(19) United States
(12) Patent Application Publication
(10) Pub. No.: US 2015/0282775 A1
(43) Pub. Date: Oct. 8, 2015

(54) C-TYPE ARM DEVICE, C-TYPE ARM HOLDING DEVICE AND X-RAY DIAGNOSTIC DEVICE

(71) Applicants: KABUSHIKI KAISHA TOSHIBA, Minato-ku (JP); Toshiba Medical Systems Corporation, Otawara-shi (JP)

(72) Inventor: Liu WENQIANG, Dalian (CN)

(73) Assignees: KABUSHIKI KAISHA TOSHIBA, Minato-ku (JP); Toshiba Medical Systems Corporation, Otawara-shi (JP)

(21) Appl. No.: 14/662,695

(22) Filed: Mar. 19, 2015

(30) Foreign Application Priority Data
Apr. 4, 2014 (CN) 201410135971.7

(51) Int. Cl. A61B 6/00; A61B 6/02

(52) U.S. Cl. CPC A61B 6/4441; A61B 6/02

(57) ABSTRACT

The present invention proposes a C-type arm device which has improved operational and rotational performance, a relevant C-type arm holding device and an X-ray diagnostic device employing said C-type arm holding device. The inventive C-type arm device comprises a base, a cantilever bracket, a C-type arm holding part and a C-type arm. The cantilever bracket is provided on the base at one end. The C-type arm holding part is provided on the other end of the cantilever bracket and is connected to it in such a way that the part is rotatable about a vertical axis. Said C-type arm and said C-type arm holding part are connected to each other in such a way that the arm is slidable along a circular arc of itself.

### FIELD OF THE INVENTION

The present invention relates to a C-type arm device, a C-type arm holding device and an X-ray diagnostic device having said C-type holding device.

### BACKGROUND

At present, medical apparatus, in which a patient on a diagnostic bed is examined by a C-type arm, are widely used. For example, with the development of computer technology, medical image diagnostic devices such as X-ray diagnostic devices, Magnetic Resonance Imaging (MRI) devices or the like become important medical imaging diagnostic apparatus.

A typical C-type arm device is a floor installed C-type arm device in which a diagnostic device having the C-type arm is installed securely on the floor.

FIG. 13 shows the schematic configuration of a known floor installed C-type arm device. As illustrated, the known C-type arm device generally comprises a struct-shaped base 100 which is installed rotatably on the floor, a main holder 200, and a C-type arm which is connected to and supported by the main holder 200.

When the illustrated C-type arm is operated, the C-type arm should be rotated manually to a position in which the C-type arm is opposite a desired area of an object to be examined and carried on the diagnostic bed, which rotating process will be called as “parking” below. In the known C-type arm device, the base 100 is embodied as a rotational center such that an operator can arduously pull at least a part of the main holder 200 to rotate it and thus to accomplish the parking. In this case, the rotational center is disposed at the lowest part of the strut-shaped base of the device.

However, when the operator carries out said parking, he/she should manually rotate both the main holder 200 and the C-type arm at the same time. Because the base undergoes the total weight of the diagnostic device via its bearings, the main holder and the C-type arm can be rotated only with a great force. Since most of medical staffs that operate the device in hospitals are females, it is inconvenient for them to carry out such operation. With respect to a relatively heavy diagnostic device having the holder and the arm, its operational performance is very poor.

Furthermore, in the known C-type arm diagnostic device, the rotational center is usually provided at the base, especially at the lowest part thereof such that when the device is required to be rotated, the main holder and the C-type arm should be rotated as a whole. Therefore, a relatively large space around the device is required to rotate the C-type arm device.

### SUMMARY OF THE INVENTION

The present invention is aimed at solving the problems mentioned above. It is an object of the present invention to provide a C-type arm device which has improved operational and rotational performances, a relevant C-type arm holding device and an X-ray diagnostic device employing said C-type arm holding device.

In one technical solution of the present invention, the C-type arm device comprises a base, a cantilever bracket, a C-type arm holding part and a C-type arm. One end of the cantilever bracket is provided on the base. The C-type arm holding part is provided to the other end of the cantilever bracket and is connected to it in such a way that the part is rotatable about a vertical axis. The C-type arm and the C-type arm holding part are connected to each other in such a way that the arm is slidable along a circular arc described by itself.

Further, in another technical solution of the present invention, the C-type arm holding device comprises a base, a cantilever bracket and a C-type arm holding part, wherein one end of the cantilever bracket is provided on the base and the other end thereof is connected to the C-type arm holding part in such a way that the bracket is rotatable about a vertical axis, the C-type arm holding part is provided to the other end of the cantilever bracket and is connected to it in such a way that the C-type arm is slidable along a circular arc described by itself.

Further, in another technical solution of the present invention, the X-ray diagnostic device comprises a base, a cantilever bracket, a C-type arm holding part and a C-type arm, wherein one end of the cantilever bracket is provided on the base, the C-type arm holding part is provided on the other end of the cantilever bracket and is connected to it in such a way that the part is rotatable about a vertical axis, the C-type arm is connected to the C-type arm holding part in such a way that the arm is slidable along a circular arc described by itself, and an X-ray generating component and an X-ray detecting component are installed on the C-type arm such that the components are opposed to each other.

According to the present invention, a rotational center is provided at the C-type arm holding part, rather than the base, such that the relevant moving parts are light weight and can be driven with a less force. Therefore, rotation is easily achieved and operational performance is improved.

Furthermore, it required in the present invention to rotate only a part of the base and the bracket such that the rotation can be achieved with a less force in an angular range and at the same time the operational performance of the whole device is improved. Further, the space required for rotation is diminished so as to facilitate rotating the device.

### BRIEF DESCRIPTION OF THE DRAWINGS

FIG. 1 is a block diagram schematically showing an entire X-ray diagnostic device according to the present invention;

FIG. 2 is a schematic view showing an entire C-type arm device according to a first embodiment;

FIG. 3 is a schematic view showing how a cantilever bracket is connected to a C-type arm holding part according to the first embodiment;

FIG. 4 is an exploded view schematically showing how the cantilever bracket is connected to the C-type arm holding part according to the first embodiment;

FIG. 5 is a structural view schematically showing a connecting shaft between the C-type arm holding part and the cantilever bracket according to the first embodiment;

FIG. 6 is a view schematically showing how the C-type arm device according to the first embodiment is operated;

FIG. 7 is a structural view schematically showing how the C-type arm holding part is connected to the C-type arm according to the first embodiment;

FIG. 8 is an exploded structural view schematically showing how the C-type arm holding part is connected to the C-type arm according to the first embodiment;

FIG. 9 is a view schematically shown how the C-type arm is slid relative to the C-type arm holding part according to the first embodiment;

FIG. 10 is a structural view schematically showing a whole structure of a C-type arm device according to a second embodiment;

FIG. 11 is a structural view schematically showing how a cantilever bracket is connected to a C-type arm holding part according to a variant example;

FIG. 12 is a structural view schematically showing how a cantilever bracket is connected to a C-type arm holding part according to another variant example; and

FIG. 13 is a structural view schematically showing a C-type arm device according to the prior art.

### A LIST OF NUMERAL REFERENCES

1. base; 2. cantilever bracket; 3. C-type arm holding part; 4. C-type art; 5. bearing holder; 6. rotational sleeve; 7. end cap; 8. rotational shaft; 9. swivel bearing; 16. hinge pivot; 11. connecting plate; 12, 13, 14, 15. sheet

### DETAILED DESCRIPTION OF THE INVENTION

The present invention relates to a C-type arm device which is applicable to various medical devices employing it. For illustrative purposes only, a floor installed X-ray diagnostic device will be explained as an example in which the C-type arm device according to the present invention is applied. However, it should be understood that the inventive C-type arm device is not limited by this specific example and can be applied in any other medical devices which are required to employ a C-type arm.

Now, preferred embodiments of the present invention will be explained with respect to the attached drawings below.

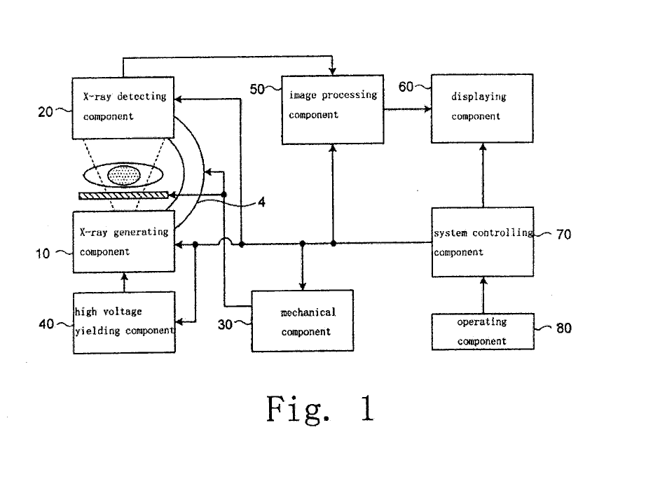

FIG. 1 is a block diagram schematically showing a whole structure of the X-ray diagnostic device involving in the present invention.

As shown in FIG. 1, the X-ray diagnostic device comprises a C-type arm 4, an X-ray generating component 10, an X-ray detecting component 20, a mechanical component 30, a high voltage yielding component 40, an image processing component 50, a displaying component 60, an operating component 80 and a system controlling component 70.

The X-ray generating component 10 and the X-ray detecting component 20 are provided on the C-type arm 4 in such a way that the components are opposed to each other. Therefore, a diagnostic bed, on which an object to be examined is carried, can be placed between the X-ray generating component 10 and the X-ray detecting component 20. The X-ray generating component 10 is provided to generate X-ray which irradiates the object to be examined on the diagnostic bed. The X-ray detecting component 20 is provided to detect the X-ray already passing through the object to be examined and generate X-ray image data.

The mechanical component 30 is a component enabling the C-type arm 4 and the diagnostic bed to be moved. The high voltage yielding component 40 supplies high voltage for the X-ray generating component 10 to generate the X-ray. The displaying component 60 is provided to display images such as X-ray images thereon. The operating component 80 is substantially constituted by a mouse or a keyboard, a joystick or the like, and is a console for acceptance of operation of an operator. The system controlling component 70 is a component which is provided to control the whole X-ray diagnostic device based on the operation of the operator. Further, the image processing component 50 is a processing component which is provided to generate an X-ray image depending on the X-ray image date generated by the X-ray detecting component 20.

The mechanical component 20, the high voltage yielding component 40, the image processing component 50, the displaying component 60, the operating component 80 and the system controlling component 70 are able to be provided in a frame of the C-type arm or alternatively in other locations thereof.

The X-ray generating component 10 and the X-ray detecting component 20 are provided on the C-type arm 4 in such a way that the components are opposed to each other. Therefore, a C-type arm holding device is provided to support the C-type arm 4 and enable it to be rotated such that the C-type arm 4 can be flexibly rotated to a location in which the arm is opposite an interesting area of the object to be examined, in which location the X-ray generating component 10 and the X-ray detecting component 20 can be operated.

Now, preferred embodiments of the C-type arm device constituted by the C-type arm holding device and the C-type arm will be explained by referring to the attached drawings below. In those embodiments, the same features are indicated by the same reference numerals and thus their repeated explanation will be omitted.

### First Embodiment

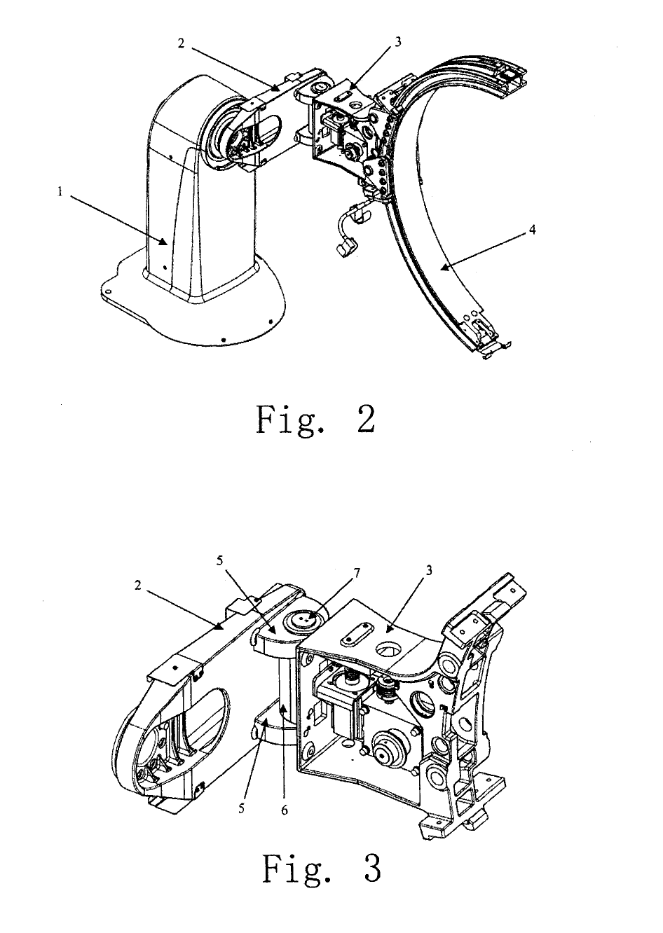

FIG. 2 is a structural view generally showing the C-type arm device according to a first embodiment. As shown in FIG. 2, the C-type arm device generally comprises a base 1 secured on the floor, a cantilever bracket 2, a C-type arm holding part 3, and a C-type arm 4, wherein one end of the cantilever bracket 2 is provided on the base 1, the C-type arm holding part 3 is provided on and connected to the other end of the cantilever bracket 2 in such a way that it is rotatable about a vertical axis with respect to the cantilever bracket 2, and the C-type arm 4 is connected to the C-type arm holding part 3 in such a way that it is slidable along the circular arc of the arm itself.

The base 1, the cantilever bracket 2, the C-type arm holding part 3 and the C-type arm 4 could be configured respectively in the same way as those in a known X-ray diagnostic device and thus their specific structures will not be explained here. In the specification, only connecting relationships between them will be explained in detail.

As shown in FIG. 2, the base 1 is provided securely on the floor in such a way it cannot be rotated.

One end of the cantilever bracket 2 is connected pivotally to the base 1 in such a way that the cantilever bracket is rotatable about a horizontal axis at its connecting location with the base. Further, the other end of the cantilever bracket 2 is connected to the C-type arm holding part 3 by a rotational assembly in such a way that the C-type arm holding part 3 is rotatable about the vertical axis while the rotational assembly being embodied as a rotating center.

The C-type arm holding part 3 is a part used to hold the C-type arm and connected directly to the C-type arm 4. Moreover, using a rack between the C-type arm holding part 3 and the C-type arm 4, the C-type arm 4 is slidable along a circular arc path described by the C-type arm itself.

The diagnostic bed carrying the object to be examined thereon is able to pass through the opening of the C-type arm 4, at which the X-ray generating component 10 (not shown in FIG. 2) and the X-ray detecting component 20 (not shown in FIG. 2) are mounted in such a way that the components are opposed to each other as shown in FIG. 1. Therefore, the X-ray generating component 10 is provided to generate X-ray towards the object to be examined, and the X-ray is detected by the X-ray detecting component 20 to obtain an X-ray image.

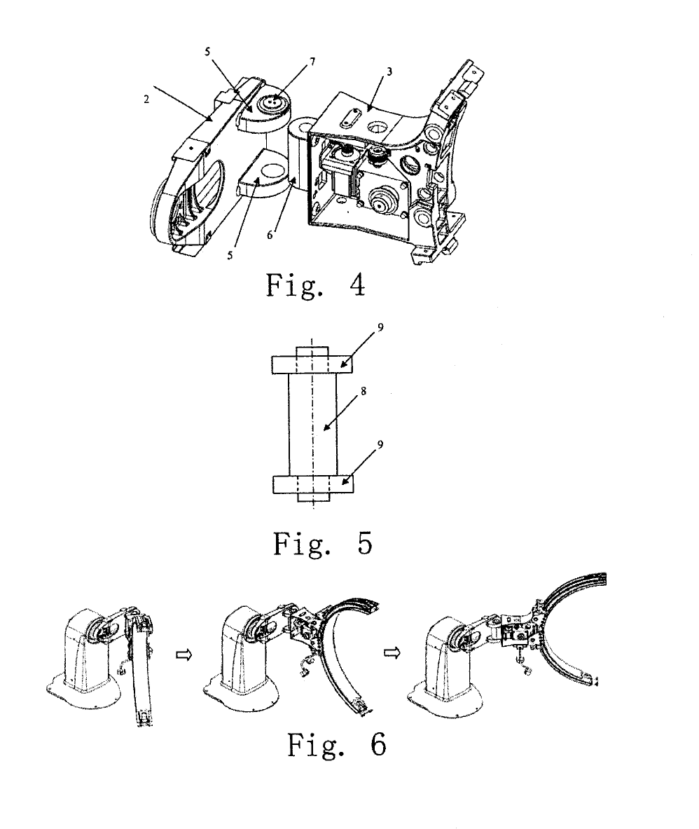

FIG. 3 is a structural view schematically showing the connecting structure (the rotational assembly) between the C-type arm holding part 3 and the cantilever bracket 2 according to the first embodiment. FIG. 4 is an exploded structural view schematically showing the connecting structure (the rotational assembly) between the C-type arm holding part 3 and the cantilever bracket 2 according to the first embodiment. FIG. 5 is a structural view schematically showing a connecting shaft between the C-type arm holding part 3 and the cantilever bracket 2 according to the first embodiment.

As shown in FIGS. 3 and 4, the rotational assembly between the C-type arm holding part 3 and the cantilever bracket 2 comprises a bearing holder 5 disposed on the cantilever bracket 2, a rotational sleeve 6 disposed on the C-type arm holding part 3, a end cap 7, a rotational shaft 8 and a swivel bearing 9.

The bearing holder 5 is secured on the cantilever bracket 2 via welding, screwing or the like. As show in FIG. 4, the bearing holder 5 has apertures for receiving the swivel bearing 9 therein. Furthermore, the rotational sleeve 6 is secured on the C-type arm holding part 3 via welding, screwing or the like. As shown in FIG. 4, the rotational sleeve 6 has an aperture for receiving the rotational shaft 8 therein.

The rotational shaft 8 is a stepped shaft having a large diameter section whose diameter corresponds to the diameter of the rotational sleeve 6 and a small diameter section whose diameter corresponds to the inner diameter of an aperture of the swivel bearing 9. The swivel bearing 9 is a hollow cylindrical structure whose diameter is larger than the rotational shaft 8 and which has an aperture matable with the small diameter section of the rotational shaft 8.

When the C-type arm holding part 3 and the cantilever bracket 2 are mounted and connected, the rotational sleeve 6 is first inserted into two bearing holders 5 such that the apertures of the bearing holders 5 communicate with the aperture of the rotational sleeve 6 The diameter of the aperture of the bearing holder 5 is larger than that of the aperture of the rotational sleeve 6.

Then, the rotational shaft 8 is inserted to pass through the apertures of the bearing holders 5 and the aperture of the rotational sleeve 6 and, subsequently, two swivel bearings 9 are mounted onto upper and lower small diameter sections of the rotational shaft 8 respectively such that the rotational shaft 8 is rotatable only in the rotational sleeve 6.

Finally, two end caps 7 are mounted securely onto the upper and lower bearing holders 5 respectively. Securing between them can be achieved in any suitable manners. In this way, the C-type arm holding part 3 is connected pivotally to the cantilever bracket 2 such that the C-type arm holding part 3 is rotatable about the vertical axis in such a way that the rotational shaft 8 is embodied as the rotational axis.

It should be noted that the connecting structure is not limited by the already mentioned contents. For example, the rotational shaft can be alternatively fabricated as a hollow cylindrical shaft which is matable directly with the rotational sleeve and whose position is limited by the end caps 7.

Alternatively, the swivel bearing 9 can be omitted. In this case, an aperture can be opened in the bearing holder 5 to mate with an end of the rotational shaft 8. That is to say, the bearing holder 5 itself is embodied as a bearing. Without consideration of wearing, those alternative structures can be used to achieve the pivoting connection between the C-type arm holding part 3 and the cantilever bracket 2.

Using the rotational assembly illustrated in FIGS. 3 to 5, the C-type arm holding part 3 is freely rotatable about the vertical axis relative to the cantilever bracket 2 in an angular range of 180 degrees as shown in FIG. 6. FIG. 6 is a view schematically showing how the C-type arm device according to the first embodiment is operated.

As shown in FIG. 6, using the rotational assembly between the C-type arm holding part 3 and the cantilever bracket 2 exclusively, the C-type arm can be rotated freely and flexibly in a relatively large angular range. Moreover, only the C-type arm suffers rotating loads such that it is more flexible than the one according to the prior art and can be operated more conveniently.

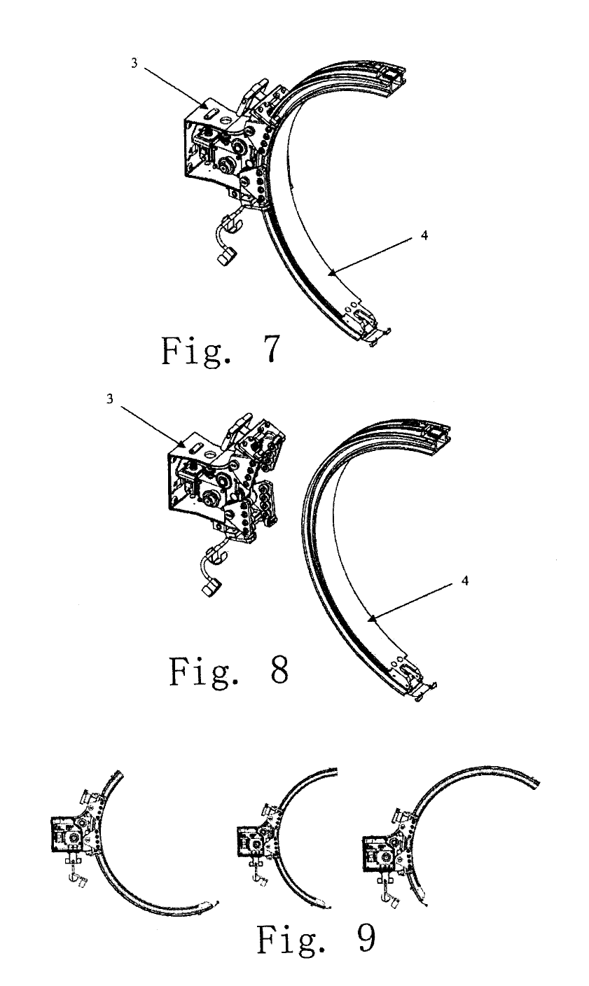

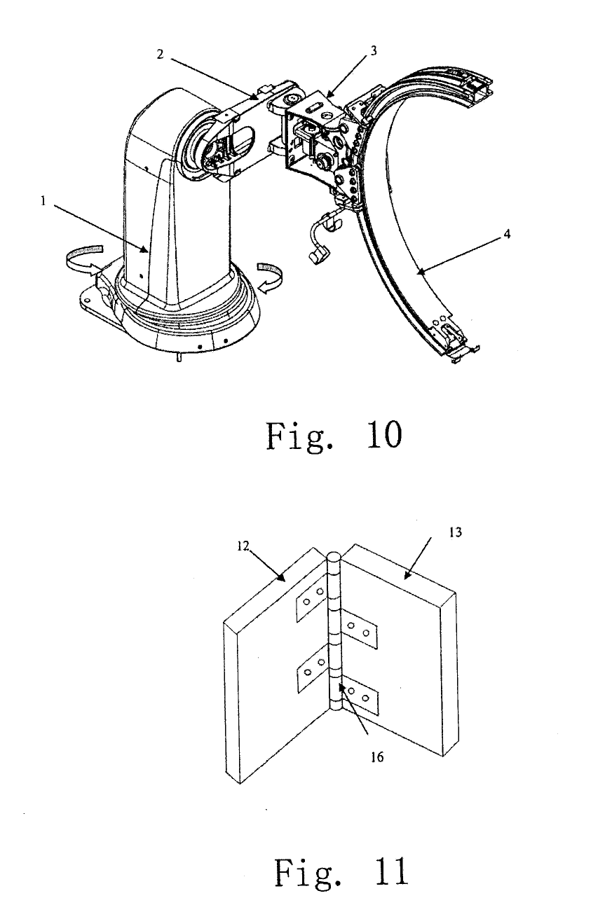

Furthermore, the C-type arm 4 is slidable along the circular arc path described by itself using the rack between the C-type arm holding part 3 and the C-type arm 4. FIG. 7 is a structural view schematically showing a connecting structure (sliding assembly) between the C-type arm holding part and the C-type arm according to the first embodiment. FIG. 8 is an exploded structural view schematically showing the connecting structure (sliding assembly) between the C-type arm holding part and the C-type arm according to the first embodiment. FIG. 9 is a view schematically showing how the connecting structure (sliding assembly) between the C-type arm holding part and the C-type arm according to the first embodiment is operated.

As shown in FIGS. 7 and 8, the rack in the form of a circular arc is provided on an edge of the C-type arm 4. Additionally, the C-type arm holding part 3 has two rows of balls at its forward end, which balls can be engaged into the rack of the C-type arm 4. Because of engagement between the balls and the rack, the C-type arm 4 can be slid along the circular arc in the range of the rack as shown in FIG. 9. In this way, the X-ray generating component 10 and the X-ray detecting component 20 on the C-type arm 4 can be re-positioned such that X-ray can be emitted towards the object to be examined and detected at different angular positions.

Additionally, a fixing device can be provided to fix the C-type arm holding part 3 in place after it is slid at a given position. For example, the C-type arm holding part 3 can be locked relative to the C-type arm 4 by fastening the balls or increasing friction between them.

According to the C-type arm device of the present invention, the rotating center is not provided at the base but at the C-type arm holding part such that relevant moving parts are made light weight. Furthermore, it is estimated that the inventive C-type arm device can be driven by a force being only 70% of the one used to drive the entire base as which the rotational center is provided. Therefore, the C-type arm can be rotated easily and its manual operational performance is improved. Particularly, the space around the device required for achieving rotation can be diminished such that the device becomes compact. Therefore, in comparison with the known device, the inventive C-type arm device can save required space.

### Second Embodiment

FIG. 10 is a structural view schematically showing an entire C-type arm device according to a second embodiment.

As shown in FIG. 10, the C-type arm device generally comprises: a base 10 installed on the floor and adapted to be rotated about a rotational axis, a cantilever bracket 2 connected the base 1 at one end, a C-type arm holding part 3 which is provided on and connected at the other end of the cantilever bracket 2 and can be rotated about a vertical axis, and a C-type arm 4 which is connected to the C-type arm holding part 3 in such a way that the arm is slidable along a circular arc described by itself.

The base 1 has a turntable under its strut, by which turntable the base can be rotated about the vertical axis while the strut being embodied as the rotating center. In this way, an enlarged rotating range can be achieved. Furthermore, a mechanism for locking the turntable can be provided such that the base can be locked when it is unnecessary to rotate the base.

The second embodiment is distinguished from the first one in that the base 10 of the second embodiment is also rotatable about the vertical axis. Other contents of the second embodiment are similar with those of the first embodiment and thus their repetitive explanations are omitted here.

Furthermore, in the second embodiment, the base 10 is rotatable by the turntable. Alternatively, the base can be rotated by means of other mechanisms allowing its rotation about the vertical axis. Alternatively, the inventive base 10 may be configured not in the form of the illustrated stud but in the form of any other shapes enabling the base to be rotated adjacent the floor.

In the second embodiment, besides the C-type arm being able to be rotatable at the C-type arm holding part 3, the C-type arm of the C-type arm device is rotatable at the base 1 such that the C-type arm device can be rotated in an increasing angular range. Rotation of the base and rotation of the C-type arm holding part are matable with each other so as to enable parking.

### Variant Examples

Now, variant examples of the present invention will be described below.

In the first and second embodiments, the rotational assembly between the C-type arm holding part 3 and the cantilever bracket 2 is achieved by a pivoting structure. However, other-type rotational assemblies can be used to achieve the rotating connection between the C-type arm holding part 3 and the cantilever bracket 2.

For instance, FIG. 11 is a structural view schematically showing a variant example of the connecting structure between the C-type arm holding part and the cantilever bracket. As shown in FIG. 11, a hinge rotational assembly is used to be connected between the C-type arm holding part 3 and the cantilever bracket 2. In the assembly, two sheets 12 and 13 are secured on the C-type arm holding part 3 and the cantilever bracket 2 respectively, and a hinge pivot 16 is provided to connect the sheets 12 and 13 so as to achieve rotation between the sheets 12 and 13 about the vertical axis. In this way, the C-type arm holding part 3 can be rotated relative to the cantilever bracket 2.

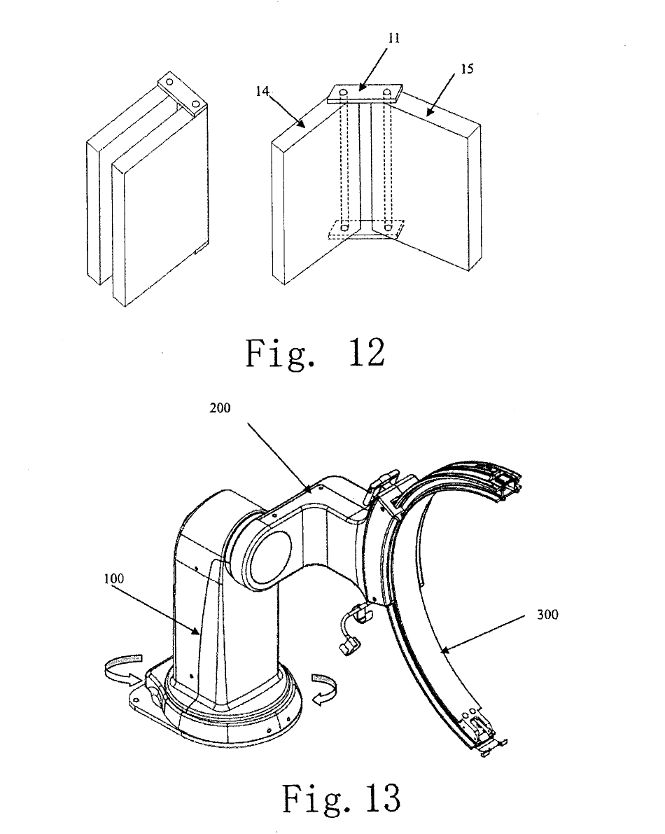

Further, FIG. 12 is a structural view schematically showing another variant example of the connecting structure between the C-type arm holding part and the cantilever bracket. As shown in FIG. 12, a rotational assembly having two pivots is provided to connect the C-type arm holding part 3 and the cantilever bracket 2, in which assembly the pivots are provided on adjacent sides of sheets 14 and 15 respectively and are connected to each other in the sheets 14 and 15 by upper and lower connecting plates 11. Therefore, a mechanism is obtained to allow a relatively large rotation arrange. In this way, the rotation between the C-type arm holding part 3 and the cantilever bracket 2 can be achieved.

Although several examples of the rotational assembly are described in the description, the present invention is not limited by them. It is perceived that other-type rotational assemblies can be used to achieve the rotation between C-type arm holding part 3 and the cantilever bracket 2. For example, provided at one end of the C-type arm holding part 3 adjacent the cantilever bracket 2 is a rack which can rotated 360 degrees about the other end of the cantilever bracket 2 so as to allow a relatively large rotational range.

Further, the sliding structure between the C-type arm holding part 3 and the C-type arm 2 is not limited by the balls of the example but can be achieved by slides, double rack engagement or the like if they can be adopted in the present invention to enable the C-type arm to be slid along the circular arc.

Although the present invention is explained with respect to several embodiments, they are presented as examples only and cannot be used to limit the scope of the present invention. The present invention can be implemented in some ways other than the already mentioned embodiments. Various alternations, changes or modifications without departing from the spirit of the present invention can be adopted. Those embodiments and their variants are included in the scope and content of the present invention and also included in the content of claims and its equivalents.

1. A C-type arm device comprising:
a base;
a cantilever bracket which is provided on the base at one end;
a C-type arm holding part which is connected to the other end of the cantilever bracket in such a way that the part is rotatable about a vertical axis; and
a C-type arm which is connected to the C-type arm holding part in such a way that it is slidable along a circular arc of the arm.

2. The C-type arm device as claimed in claim 1, wherein the cantilever bracket is provided such that it is rotatable about a horizontal axis at its connecting location with the base.

3. The C-type arm device as claimed in claim 1, wherein the other end of the cantilever bracket is connected to the C-type arm holding part using a bearing component in a direction of the vertical axis.

4. The C-type arm device as claimed in claim 1, wherein the base is provided in such a way that it is rotatable about the vertical axis.

5. A C-type arm holding device comprising:
a base;
a cantilever bracket which is provided on the base at one end and is connected with a C-type arm holding part at the other end in such a way that the cantilever bracket is rotatable about a vertical axis; and
the C-type arm holding part which is provided at the other end of the cantilever bracket and is connected to a C-type arm in such a way that the C-type arm is slidable along a circular arc of the arm itself.

6. An X-ray diagnostic device comprising:
a base;
a cantilever bracket which is provided on the base at one end;
a C-type arm holding part which is provided to the other end of the cantilever bracket and is connected to the cantilever bracket in such a way that the part is rotatable about a vertical axis; and
a C-type arm which is connected to the C-type arm holding part in such a way that the C-type arm is slidable along a circular arc of the arm itself, wherein an X-ray generating component and an X-ray detecting component are provided on the C-type arm in such a way that the components are opposed to each other.
